(19) United States
(12) Patent Application Publication
(10) Pub. No.: US 2014/0107130 A1
(43) Pub. Date: Apr. 17, 2014

(54) Oral Solution Formulations of Aripiprazole

(71) Applicant: Antrim Pharmaceuticals, LLC, Lake Forest, IL (US)

(72) Inventors: Brian Tambi, Lake Forest, IL (US); David Song, Lake Forest, IL (US)

(73) Assignee: Antrim Pharmaceuticals, LLC, Lake Forest, IL (US)

(21) Appl. No.: 14/052,517

(22) Filed: Oct. 11, 2013

Related U.S. Application Data
(60) Provisional application No. 61/712,461, filed on Oct. 11, 2012.

(51) Int. Cl. A61K 47/22; A61K 31/496

(52) U.S. Cl. CPC A61K 47/22; A61K 31/496
USPC 514/253.07

(57) ABSTRACT

Oral solution formulations of aripiprazole, methods of manufacture of these formulations, methods of administration, and kits containing same.

### FIELD OF THE INVENTION

The present invention relates to oral solution aripiprazole formulations which are suitable for long-term storage, methods of manufacture of the formulations, methods of their administration, and kits containing the same.

### BACKGROUND OF THE INVENTION

Aripiprazole is a partial dopamine agonist of the third generation class of atypical antipsychotics with additional antidepressant properties that is used in the treatment of schizophrenia, bipolar disorder, and clinical depression. It is marketed in the United States as Abilify®.

It has the following structure:

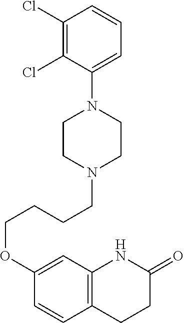 [SMILES: O=C1CCc2ccc(OCCCCN3CCN(c4cccc(Cl)c4Cl)CC3)cc2N1]

Aripiprazole has poor aqueous solubility (<1 μg/mL at room temperature). When formulated as an intramuscular (IM) injectable solution, aripiprazole has been found to cause unacceptable (moderate to severe) tissue irritation at the muscular site with many water-miscible co-solvent systems, and water-immiscible solvent and co-solvent systems such as hexonoic acid:medium chain triglyceride (10:90), polyethylene glycol 400:ethanol:lactic acid (35:15:50), benzyl alcohol:sesame oil (10:90), benzyl alcohol:medium chain triglyceride (10:90), benzyl alcohol:tributyrin (5:95), and polysorbate 80 in 25 mM tartaric acid.

While oral solution formulations of aripiprazole are known (see, for example, U.S. Pat. No. 6,977,257 B2), they generally include sugars and/or sugar alcohols, such as sorbitol. It would be desirable to arrive at sugar-free formulations which would be suitable for use by, for example, diabetic patients. It would also be desirable to avoid the need for use of a preservative in the oral solution formulations. In addition, the compositions should be suitably stable for long term storage without substantial loss of effectiveness.

### SUMMARY OF THE INVENTION

The invention provides stable oral solution sugar-free formulations of aripiprazole that allow its long term storage.

In one embodiment, the invention provides a pharmaceutical formulation suitable for oral administration comprising aripiprazole, glycerin, propylene glycol, a buffer, a sweetener, and a flavoring agent, wherein said pharmaceutical formulation does not comprise a sugar, and wherein said pharmaceutical formulation has a pH of 4.3 or greater.

In a preferred embodiment, the pharmaceutical formulation does not comprise a preservative.

In a preferred embodiment, the sweetener is saccharin. Other suitable sweeteners include, but are not limited to, aspartame, cyclamate, stevia, sucralose, and others.

In another preferred embodiment, the buffer comprises citric acid and sodium citrate. It is within the skill of the art to determine the appropriate buffer.

In a preferred embodiment, the pharmaceutical formulation has a pH of about 4.5.

In one embodiment, the aripiprazole is present at a concentration of about 0.5 mg/ml to about 1.5 mg/ml; more preferably at about 1.0 mg/ml.

In one embodiment, the glycerin is present at a concentration of about 30% to about 70%; more preferably at about 50% to about 60%; and most preferably at about 50%.

In one embodiment, the propylene glycol (PG) is present at a concentration of about 15% to about 30%; and most preferably at about 20%.

In one preferred embodiment, the invention provides a preservative-free pharmaceutical formulation suitable for oral administration comprising aripiprazole at a concentration of about 0.5 mg/ml to about 1.5 mg/ml, glycerin at a concentration of about 30% to about 70%, propylene glycol at a concentration of about 15% to about 30%, citric acid, sodium citrate, saccharin, and a flavoring agent, wherein said pharmaceutical formulation does not comprise a sugar, and wherein said pharmaceutical formulation has a pH of about 4.3 or greater.

In another preferred embodiment, the invention provides a preservative-free pharmaceutical formulation suitable for oral administration comprising aripiprazole at a concentration of about 1.0 mg/ml, glycerin at a concentration of about 50%, propylene glycol at a concentration of about 25% citric acid, sodium citrate, saccharin, and a flavoring agent, wherein said pharmaceutical formulation does not comprise a sugar, and wherein said pharmaceutical formulation has a pH of about 4.5.

In yet another embodiment, the invention provides a method of treating schizophrenia comprising administering to a patient in need thereof a therapeutically effective amount of one of the pharmaceutical formulations of the invention.

These and other aspects will become apparent from the following description of the various embodiments, although variations and modifications therein may be affected without departing from the spirit and scope of the novel concepts of the disclosure.

It is to be understood that both the foregoing general description and the following detailed description are exemplary and explanatory only and are not restrictive of the invention, as claimed.

### DETAILED DESCRIPTION OF THE INVENTION

Various embodiments of the invention are now described in detail. As used in the description and throughout the claims, the meaning of “a”, “an”, and “the” includes plural reference unless the context clearly dictates otherwise. Also, as used in the description and throughout the claims, the meaning of “in” includes “in” and “on” unless the context clearly dictates otherwise. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification.

Unless otherwise defined, all technical and scientific terms used herein have the same meaning as commonly understood by one of ordinary skill in the art to which this invention pertains. In the case of conflict, the present document, including definitions will control.

“Around,” “about” or “approximately” shall generally mean within 10 percent, within 5, 4, 3, 2 or 1 percent of a given value or range. Numerical quantities given are approximate, meaning that the term “around,” “about” or “approximately” can be inferred if not expressly stated.

The term “aripiprazole” is synonymous with the active pharmaceutical ingredient in Abilify®. It is a partial dopamine agonist of the third generation class of atypical antipsychotics with additional antidepressant properties that is used in the treatment of schizophrenia, bipolar disorder, and clinical depression. For the purposes of the present application, the term “aripiprazole” also encompasses pharmaceutically acceptable salts of aripiprazole.

The term “sugar” refers to monosaccharides, disachharides, and polysaccharides. Examples of sugars include, but are not limited to, sucrose, glucose, dextrose, and others.

The term “flavoring agent” refers to any agent which affects the flavor of the provided compositions. Examples of flavoring agents include, but are not limited to, natural flavoring substances, nature-identical flavoring substances and artificial flavoring substances.

The term “long-term storage” is understood to mean that the pharmaceutical composition can be stored for three months or more, for six months or more, and preferably for one year or more. Long-term storage is also understood to mean that the pharmaceutical composition is stored either as a liquid at 2-8° C., or is frozen, e.g., at −20° C., or colder. It is also contemplated that the composition can be frozen and thawed more than once.

The term “stable” with respect to long-term storage is understood to mean that aripiprazole contained in the pharmaceutical compositions does not lose more than 20%, or more preferably 15%, or even more preferably 10%, and most preferably 5% of its activity relative to activity of the composition at the beginning of storage.

The term “substantially free” means that either no substance is present or only minimal, trace amounts of the substance are present which do not have any substantial impact on the properties of the composition. In one embodiment, no amount of a substance includes “no detectable amount”.

The term “mammal” includes, but is not limited to, a human.

The term “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

The terms “pharmaceutical composition” and “formulation” are used interchangeably.

The term “treatment” refers to any administration or application of remedies for disease in a mammal and includes inhibiting the disease, arresting its development, relieving the disease, for example, by causing regression, or restoring or repairing a lost, missing, or defective function; or stimulating an inefficient process. The term includes obtaining a desired pharmacologic and/or physiologic effect, covering any treatment of a pathological condition or disorder in a mammal. The effect may be prophylactic in terms of completely or partially preventing a disorder or symptom thereof and/or may be therapeutic in terms of a partial or complete cure for a disorder and/or adverse affect attributable to the disorder. It includes (1) preventing the disorder from occurring or recurring in a subject who may be predisposed to the disorder but is not yet symptomatic, (2) inhibiting the disorder, such as arresting its development, (3) stopping or terminating the disorder or at least its associated symptoms, so that the host no longer suffers from the disorder or its symptoms, such as causing regression of the disorder or its symptoms, for example, by restoring or repairing a lost, missing or defective function, or stimulating an inefficient process, or (4) relieving, alleviating or ameliorating the disorder, or symptoms associated therewith, where ameliorating is used in a broad sense to refer to at least a reduction in the magnitude of a parameter, such as inflammation and/or pain.

The term “disease” refers to any condition, infection, disorder or syndrome that requires medical intervention or for which medical intervention is desirable. Such medical intervention can include treatment, diagnosis and/or prevention.

The term “therapeutically effective amount” refers to an amount which, when administered to a living subject, achieves a desired effect on the living subject. For example, an effective amount of the pharmaceutical compositions of the invention for administration to the living subject is an amount that prevents and/or treats a disease treatable with aripiprazole, for example, schizophrenia. The exact amount will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art.

### Embodiments of the Invention

### Aripiprazole

All of the compositions of the present invention comprise aripiprazole. As explained in the Background section of this application, aripiprazole is a partial dopamine agonist of the third generation class of atypical antipsychotics with additional antidepressant properties that is used in the treatment of schizophrenia, bipolar disorder, and clinical depression. It is marketed in the United States as Abilify®.

It has the following structure:

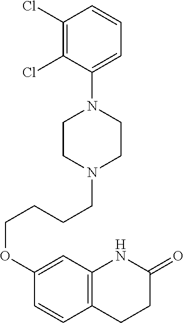 [SMILES: O=C1CCc2ccc(OCCCCN3CCN(c4cccc(Cl)c4Cl)CC3)cc2N1]

Aripiprazole has been described, for example, in U.S. Pat. Nos. 4,734,416 and 5,006,528.

### Oral Formulations

In one embodiment, the invention provides a pharmaceutical formulation suitable for oral administration comprising aripiprazole, glycerin, propylene glycol, a buffer, a sweetener, and a flavoring agent, wherein said pharmaceutical formulation does not comprise a sugar, and wherein said pharmaceutical formulation has a pH of 4.3 or greater.

The term “pH of 4.3 or greater” specifically excludes all formulations which have a pH of between 4.0 and less than 4.3.

In a preferred embodiment, the pharmaceutical formulation does not comprise a preservative.

In a preferred embodiment, the sweetener is saccharin. Other suitable sweeteners include, but are not limited to, aspartame, cyclamate, stevia, sucralose, and others.

In another preferred embodiment, the buffer comprises citric acid and sodium citrate. While other buffers may potentially be used, it was found that tartaric buffer system causes significant precipitation of aripiprazole when pH is about 4.7. Accordingly, in preferred embodiments, the compositions of the invention do not comprise tartaric buffer. It was also found that the compositions comprising acetic acid/sodium acetate buffer was physically stable over about 6 days in refrigerator (2-8° C.). It is within the skill of the art to determine the appropriate buffer.

In a preferred embodiment, the pharmaceutical formulation has a pH of about 4.5.

In one embodiment, the aripiprazole is present at a concentration of about 0.5 mg/ml to about 1.5 mg/ml; more preferably at about 1.0 mg/ml.

In one embodiment, the glycerin is present at a concentration of about 30% to about 70%; more preferably at about 50% to about 60%; and most preferably at about 50%.

In one embodiment, the propylene glycol (PG) is present at a concentration of about 15% to about 30%; and most preferably at about 20%.

These preferred concentrations of glycerin and PG are antimicrobial, which provides an additional benefit to the inventive formulations.

The pharmaceutical compositions of the invention may also include water.

In one preferred embodiment, the invention provides a preservative-free pharmaceutical formulation suitable for oral administration comprising aripiprazole at a concentration of about 0.5 mg/ml to about 1.5 mg/ml, glycerin at a concentration of about 30% to about 70%, propylene glycol at a concentration of about 15% to about 30%, citric acid, sodium citrate, saccharin, and a flavoring agent, wherein said pharmaceutical formulation does not comprise a sugar, and wherein said pharmaceutical formulation has a pH of about 4.3 or greater.

In another preferred embodiment, the invention provides a preservative-free pharmaceutical formulation suitable for oral administration comprising aripiprazole at a concentration of about 1.0 mg/ml, glycerin at a concentration of about 50%, propylene glycol at a concentration of about 25% citric acid, sodium citrate, saccharin, and a flavoring agent, wherein said pharmaceutical formulation does not comprise a sugar, and wherein said pharmaceutical formulation has a pH of about 4.5.

A person of ordinary skill in the art will understand that the combining of the various components to be included in the provided formulations can be done in any appropriate order.

The formulations of the invention may also optionally include other buffering agents, tonicity modifiers, excipients, pharmaceutically acceptable carriers, surfactants, stabilizers, colorants and other commonly used inactive ingredients of the pharmaceutical compositions.

### Additional Components of the Provided Pharmaceutical Compositions

The formulations of the invention may also include other buffers (unless they are specifically excluded in the description of the specific embodiments of the invention), tonicity modifiers, excipients, pharmaceutically acceptable carriers and other commonly used inactive ingredients of the pharmaceutical compositions.

Examples of suitable excipients include but are not limited to polymers such as: serum albumin (bovine serum albumin (BSA), human SA or recombinant HA), dextran, PVA, hydroxypropyl methylcellulose (HPMC), polyethyleneimine, gelatin, polyvinylpyrrolidone (PVP), hydroxyethylcellulose (HEC); non-aqueous solvents such as: polyhydric alcohols, (e.g., PEG, ethylene glycol and glycerol) dimethysulfoxide (DMSO) and dimethylformamide (DMF); amino acids such as: proline, L-serine, sodium glutamic acid, alanine, glycine, lysine hydrochloride, sarcosine and gamma-aminobutyric acid; surfactants such as: Tween®-80 (polysorbate 80), Tween®-20 (polysorbate 20), SDS, polysorbates, poloxamers; and miscellaneous excipients such as: potassium phosphate, sodium acetate, ammonium sulfate, magnesium sulfate, sodium sulfate, trimethylamine N-oxide, betaine, CHAPS, monolaurate, 2-O-beta-mannoglycerate or any combination of the above.

Suitable excipients, tonicity modifiers, surfactants, colorants, and other commonly used inactive ingredients may be present in the compositions of the invention unless they are specifically excluded in the description of the specific embodiments of the invention.

### Methods of Treatment

In another embodiment, the invention provides a method of treating a mammal comprising orally administering a therapeutically effective amount of the pharmaceutical compositions of the invention to a mammal, wherein the mammal has a disease or disorder that can be beneficially treated with aripiprazole.

In a preferred embodiment, the mammal is a human.

Diseases or disorders that can be treated with the provided compositions include but are not limited to schizophrenia.

In one embodiment, the invention provides a method of treatment and/or prevention of schizophrenia comprising orally administering to a mammal in need thereof a therapeutically effective amount of one of the provided aripiprazole compositions.

The therapeutically effective amount of the aripiprazole in the provided compositions will depend on the condition to be treated, the severity of the condition, prior therapy, and the patient's clinical history and response to the therapeutic agent.

The proper dose can be adjusted according to the judgment of the attending physician such that it can be administered to the patient one time or over a series of administrations.

The pharmaceutical compositions can be administered as a sole therapeutic or in combination with additional therapies as needed. Thus, in one embodiment, the provided methods of treatment and/or prevention are used in combination with administering a therapeutically effective amount of another active agent. The other active agent may be administered before, during, or after administering the pharmaceutical compositions of the present invention. Another active agent may be administered either as a part of the provided compositions, or alternatively, as a separate formulation.

The pharmaceutical compositions may, if desired, be presented in a vial, pack or dispenser device which may contain one or more unit dosage forms containing the active ingredient. In one embodiment the dispenser device can comprise a syringe having a single dose of the liquid formulation ready for injection. The syringe can be accompanied by instructions for administration.

In another embodiment, the present invention is directed to a kit or container, which contains an aqueous pharmaceutical composition of the invention. The concentration of the aripiprazole in the aqueous pharmaceutical composition can vary over a wide range, but is generally within the range of from about 0.1 to about 1.5 milligrams per milliliter (mg/ml) of aqueous formulation. The kit can also be accompanied by instructions for use.

The present invention is more particularly described in the following examples that are intended as illustrative only, since many modifications and variations therein will be apparent to those skilled in the art.

### EXAMPLE 1

### Inventive Oral Formulations of Aripiprazole

A stable pharmaceutical composition suitable for oral administration containing aripiprazole may contain the following ingredients listed in Table 1:

TABLE 1

| Ingredient | Amount per 1.0 ml | Unit |
|---|---|---|
| Aripiprazole, USP | 1.0 | mg |
| Glycerin | 450 | mg |
| Citric Acid, 1M | 86 | mg |
| Sodium Citrate | ~50 | mg |
| Propylene Glycol | 250 | mg |
| Saccharin | 4.0 | mg |
| Peach flavoring | 3 | mg |
| agent | | |
| Water | Q.S. to volume | |

The compositions can be tested for long-term stability by High Performance

Liquid Chromatography (HPLC) or other methods which are well known to skilled artisans.

It is believed that the composition will be stable over the term of two years or more.

A stable pharmaceutical composition suitable for oral administration containing aripiprazole may also contain the following ingredients listed in Table 2:

TABLE 2

| | | | Wt/ml of |
|---|---|---|---|
| Sr. | Material | | syrup |
| No. | Description | Specification | (mg) |
| 1. | Aripiprazole | IH | 1.00 |
| 2. | Glycerin | USP | 200.00 |
| 3. | Citric Acid | USP | 18.07 |
| | Monohydrate | | |
| 4. | Sodium Citrate | USP | 12.90 |
| 5. | Propylene Glycol | USP | 600.00 |
| 6. | Saccharin Sodium | USP | 4.00 |
| 7. | Peach flavoring | IH | 3.00 |
| | agent | | |
| 8. | Purified Water | USP | q.s. to |
| | | | 1 ml |

The following manufacturing procedure may be used to arrive at the formulation listed in Table 2.
1. Citric acid monohydrate USP may be dissolved in purified water to arrive at 1M solution.
2. Sodium Citrate USP may be dissolved in purified water to arrive at 1M solution.
3. Propylene Glycol USP, Aripiprazole IH and Glycerin USP may be transferred into the manufacturing tank under continuous stirring.
4. The 1 M Citric Acid Monohydrate USP solution may be added to the mixture of step 3; the resultant mixture was mixed for 60 minutes and observe for the formation of clear solution.
5. Saccharin Sodium USP may be added to the mixture of step 4 and mixed for about 15 minutes.
6. Peach flavoring agent may be added to the mixture of step 5 and mixed for about 15 minutes.
7. The 1 M Sodium citrate USP solution may be added to the mixture of step 6 and mixed for about 15 minutes.
8. The pH of the mixture of step 7 may be measured; should be between 4.30 to 4.60.
9. The volume should be adjusted to final volume and pH should be checked again; should be between 4.30 to 4.60.
10. The solution may be filtered through Polypropylene filter pads (e.g., 5-10 microns).
11. 150 mL aliquots of the resultant formulation can be filled in to round amber PET Bottles and capped, for example, using CR-Cap technology, followed by induction sealing.

### EXAMPLE 2

### Comparative Oral Solution Formulation of Aripiprazole

The purpose of this experiment was to evaluate the stability of the aripiprazole formulation which was very similar to the one disclosed in U.S. Pat. No. 6,977,257 B2, except it had a pH of about 4.6

The reference formulation of aripiprazole was prepared as follows:

First, stock solutions of methyl paraben (MP) and propyl paraben (PP) (100 mg/g in propylene glycol) were prepared.

Second, concentrated lactic buffer solutions at different pH were prepared: 100 mg/ml at pH 3.2; 100 mg/ml at pH 3.8; and 100 mg/ml at pH 4.7.

Third, EDTA stock solution (100 mg/ml in water) was prepared.

Then, the formulation was prepared as follows:
1. 15 g of glycerin was added into a beaker.
2. Then, about 15 ml of water, 847 mg of lactic acid, and 4 g of PG was added to the same beaker and mixed.
3. Then, 1 ml of stock EDTA solution and 100 mg of aripiprazole was added and mixed until clear.
4. Then, pH was adjusted to 3.1-3.2.
5. Then, the beaker was heated to about 50° C.
6. Then, about 2 g of MP/PP stock was added and mixed until clear.
7. Then, the temperature was reduced to about 45° C., 15 g of sucrose and 20 g of fructose was added and stirred until dissolved.
8. Then, the final volume was adjusted to 100 ml with water.

The final pH was then adjusted to 1.7; 2.2; 3.1; or 4.6 with either HCL or NaOH. All samples were stored in 20 ml glass vials at 40° C. or 60° C. Table 2 lists the ingredients of the compositions.

TABLE 3

| | Concentration |
|---|---|
| Ingredient | (mg/ml) |
| Aripiprazole, USP | 1.0 |
| Glycerin | 150 |
| Lactic Acid | 8.47 |
| Sodium | 0.45 |
| Hydroxide | |
| Propylene Glycol | 50 |
| EDTA | 1 |
| Methyl Paraben | 0.8 |
| Propyl Paraben | 0.08 |
| Sucrose | 150 |
| Fructose | 200 |
| Natural Orange | 3 |
| Cream Flavor | |
| Water | 436.2 |

Six days after the preparation, the samples were tested with HPLC and no significant degradation was observed at all pH levels. There was no difference observed between pH levels in chemical stability.

However, at room temperature and in refrigerator (2-8° C.), the sample at pH 4.6 showed precipitation after a few days' storage. It was therefore concluded that pH 4.6 formulation of this Example is not physically stable and will precipitate under normal storage conditions.

### EXAMPLE 3

### Solubility of Various Formulations of Aripiprazole

The purpose of this experiment was to test solubility of aripiprazole at various combinations of glycerol and propylene glycol (PG).

Formulations 1-4 consisted of acetic acid buffer system; 1 mg/ml aripiprazole; and 70% total level of glycerin and PG (glycerin plus PG=70%). The relative ratio of glycerin to PG was varied as follows:

Formulation 1: 6% PG;

Formulation 2: 10% PG;

Formulation 3: 20% PG; and

Formulation 4: 30% PG.

The final pH level of all formulations was adjusted to around 4.5.

All formulations were clear after 2 days of storage in refrigerator (2-8° C.).

Table 4 lists the ingredients of these formulations and observations after initial preparation and storage for 2 days in refrigerator.

TABLE 4

| Ingredients | Formulation 1 | | Formulation 2 | | Formulation 3 | | Formulation 4 | |
|---|---|---|---|---|---|---|---|---|
| Aripiprazole | 10.6 | mg | 10.99 | mg | 10.26 | mg | 10.22 | mg |
| PG | 0.67 | g | 1.02 | g | 2.04 | g | 2.92 | g |
| Glycerin | 6.59 | g | 6 | g | 4.97 | g | 3.91 | g |
| Acetic Acid, | 800 | mg | 800 | mg | 800 | mg | 800 | mg |
| 1M | | | | | | | | |
| Initial pH | 3.77 | | 3.84 | | 4.75 | | 4.88 | |
| Sodium | 200 | mg | 200 | mg | | | | |
| Acetate, 1M | | | | | | | | |
| Adjusted pH | 4.5 | | 4.59 | | | | | |
| Acetic Acid, | | | | | 60 | mg | 120 | mg |
| 1M | | | | | | | | |
| Adjusted pH | | | | | 4.66 | | 4.73 | |
| Water | Q.S. to 10 | ml | Q.S. to 10 | ml | Q.S. to 10 | ml | Q.S. to 10 | ml |

| | | | | |
|---|---|---|---|---|
| Initial | clear | clear | clear | clear |
| Observations | | | | |
| Observations | clear | clear | clear | clear |
| after storage | | | | |
| for 2 days at | | | | |
| 2-8° C. | | | | |

Then, the absolute solubility of aripiprazole in these formulations was tested as follows. About 5-10 ml of each formulation was removed into separate 20 ml vials, about 10 mg of aripiprazole API was added into each vial and the vials were stirred overnight.

Table 5 demonstrates the results of this experiment.

TABLE 5

| | | | Formu- | Formu- |
|---|---|---|---|---|
| | Formulation 1 | Formulation 2 | lation 3 | lation 4 |
| Overnight | cloudy | cloudy | cloudy | cloudy |
| Observations | | | | |
| Second Night | cloudy | cloudy | Partially | Partially |
| Observations | | | cloudy | cloudy |

Then, each sample was filtered with 0.2 μm centrifugal filter and was collected in a pre-weight tube. Table 6 shows the weights of the samples.

TABLE 6

| | Formu- | Formu- | | |
|---|---|---|---|---|
| | lation 1 | lation 2 | Formulation 3 | Formulation 4 |
| Weight pre- | 0.8923 | 0.88236 | 0.91504 | 0.8575 |
| filtration (mg) | | | | |
| Weight post- | 1.7076 | 1.5872 | 1.593 | 1.4841 |
| filtration (mg) | | | | |
| Filtrate weight | 0.8153 | 0.70484 | 0.67796 | 0.6266 |
| (mg) | | | | |

Then, all samples were transferred into a 25 ml flask, Q.S. with diluent (50% Acetonitrile, 0.1% FA), and HPLC was run on all samples and compared to standard (0.192 mg/ml in diluent).

The experiment demonstrated that the solubility was as follows:

PG 6%: about 1.3 mg/ml;

PG 10%: about 1.5 mg/ml;

PG 20%: about 2.6 mg/ml; and

PG 30%: about 2.5 mg/ml.

Accordingly, maximum solubility of aripiprazole was achieved at about 20% PG.

Therefore, in a preferred embodiment of the invention, the concentration of PG is about 20% and the concentration of glycerin is about 50%.

### EXAMPLE 4

### Excipient Selection Study for Various Formulations of Aripiprazole

The purpose of this experiment was to determine which additional excipients could most beneficially be added to the provided formulations.

Formulations 1, 2 and 6 consisted of the base formulations with acetic buffer, citric buffer, and lactic acid buffer systems. They did not include EDTA or fructose.

Formulation 3 was based on citric buffer system and also included EDTA.

Formulation 4 was based on citric buffer system and also included EDTA and saccharin.

Formulation 5 was based on citric buffer system and also included EDTA and fructose.

All formulations were adjusted to pH around 4.5 in the final preparation step and were all clear.

After storage at 40° C. for about 8 days, formulation 5 showed yellow color and was eliminated from the selection.

Table 7 lists the ingredients of these formulations and observations.

TABLE 7

| | Formulation 1 | | Formulation 2 | | Formulation 3 | | Formulation 4 | | Formulation 5 | | Formulation 6 | |
|---|---|---|---|---|---|---|---|---|---|---|---|---|
| Aripiprazole | 10.6 | mg | 10.99 | mg | 10.26 | mg | 10.22 | mg | 10.22 | mg | 10.22 | mg |
| PPGPG | 2 | g | 2 | g | 2 | g | 2 | g | 2 | g | 2 | g |
| Glycerin | 5 | g | 5 | g | 5 | g | 5 | g | 5 | g | 5 | g |
| Acetic Acid, | 860 | mg | | | | | | | | | | |
| 1M | | | | | | | | | | | | |
| Citric Acid, | | | 860 | μl | 860 | μl | 860 | μl | 860 | μl | | |
| 1M | | | | | | | | | | | | |
| Lactic Acid, | | | | | | | | | | | 860 | μl |
| 1M | | | | | | | | | | | | |
| Stir | | | | | | | | | | | | |

| | | | | | | |
|---|---|---|---|---|---|---|
| Observations | clear | clear | clear | clear | clear | clear |

| | | | | | | |
|---|---|---|---|---|---|---|
| EDTA, 100 mg/ml | 0.1 | ml | 0.1 | ml | 0.1 | ml |

| | |
|---|---|
| Fructose, 5.8 g/ml | Q.S. |

| | | |
|---|---|---|
| Saccharin | 0.05 | g |

| | | | | | | |
|---|---|---|---|---|---|---|
| Water | Q.S. | Q.S. | Q.S. | Q.S. | | Q.S. |
| Stir | | | | | | |
| pH | ~3.5 | ~2.7 | ~3.0 | ~2.7 | ~3.0 | ~2.8 |
| pH | 4.5 | | | | | |
| adjustment | | | | | | |
| with Sodium | | | | | | |
| Acetate, 1M | | | | | | |
| pH | | 4.5 | 4.5 | 4.5 | 4.5 | |
| adjustment | | | | | | |
| with Sodium | | | | | | |
| Citrate, 1M | | | | | | |
| pH | | | | | | 4.5 |
| adjustment | | | | | | |
| with Sodium | | | | | | |
| Hydroxide, | | | | | | |
| 1M | | | | | | |
| Observations | clear | clear | clear | clear | clear | clear |

After storage for 8 days at 40° C., HPLC analysis was performed on all six formulations, together with aripiprazole standard and Abilify® sample.

The results of the HPLC analyses demonstrated that the Abilify® sample contained methylparaben (MP) and propylparaben (PP) peaks, and formulation 4 contained saccharin peak. Formulation 5 showed differentially higher degradation peak than the other 5 formulations.

After storage for 8 days at 50° C., differential degradation patterns were observed at the tested formulations. The formulation with the lowest degradation peak was formulation 2 (which contained citric acid but did not include EDTA or fructose).

After storage for 13 days at 40° C., HPLC profiles were observed which were similar to the HPLC profiles observed after 8 days of storage at 40° C.

After storage for 13 days at 50° C., HPLC profiles were observed which were similar to the HPLC profiles observed after 8 days of storage at 50° C. Again, formulation 2 had the lowest degradation peak.

### EXAMPLE 5

### Taste Masking Study for Various Formulations of Aripiprazole

The purpose of this experiment was to determine whether saccharin and/or other flavoring agents affect stability of the formulations.

The aripiprazole formulation was prepared, containing 1 mg/ml aripiprazole, 20% PG; 50% glycerin, and citric acid buffer system at pH 4.5. Saccharin and other flavoring agents were added. The concentrations of saccharin and other flavoring agents is not essential.

The formulation was prepared similarly to the way other inventive or comparative formulations were prepared. Then, the formulation was tested for stability at 40° C. After 4 weeks of storage at 40° C., no significant degradation was observed.

The foregoing description of the exemplary embodiments of the invention has been presented only for the purposes of illustration and description and is not intended to be exhaustive or to limit the invention to the precise forms disclosed. Many modifications and variations are possible in light of the above teaching.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pharmaceutical formulation suitable for oral administration comprising aripiprazole, glycerin, propylene glycol, a buffer, a sweetener, and a flavoring agent, wherein said pharmaceutical formulation does not comprise a sugar, and wherein said pharmaceutical formulation has a pH of 4.3 or greater.

2. The pharmaceutical formulation of claim 1, wherein said pharmaceutical formulation does not comprise a preservative.

3. The pharmaceutical formulation of claim 1, wherein said sweetener is saccharin.

4. The pharmaceutical formulation of claim 1, wherein said buffer comprises citric acid and sodium citrate.

5. The pharmaceutical formulation of claim 1, wherein the pH is about 4.5.

6. The pharmaceutical formulation of claim 1, wherein said aripiprazole is present at a concentration of about 0.5 mg/ml to about 1.5 mg/ml.

7. The pharmaceutical formulation of claim 6, wherein said concentration is about 1.0 mg/ml.

8. The pharmaceutical formulation of claim 1, wherein said glycerin is present at a concentration of about 30% to about 70%.

9. The pharmaceutical formulation of claim 1, wherein said propylene glycol is present at a concentration of about 15% to about 30%.

10. A preservative-free pharmaceutical formulation suitable for oral administration comprising aripiprazole at a concentration of about 0.5 mg/ml to about 1.5 mg/ml, glycerin at a concentration of about 30% to about 70%, propylene glycol at a concentration of about 15% to about 30%, citric acid, sodium citrate, saccharin, and a flavoring agent, wherein said pharmaceutical formulation does not comprise a sugar, and wherein said pharmaceutical formulation has a pH of about 4.3 or greater.

11. A preservative-free pharmaceutical formulation suitable for oral administration comprising aripiprazole at a concentration of about 1.0 mg/ml, glycerin at a concentration of about 50%, propylene glycol at a concentration of about 25% citric acid, sodium citrate, saccharin, and a flavoring agent, wherein said pharmaceutical formulation does not comprise a sugar, and wherein said pharmaceutical formulation has a pH of about 4.5.

12. A method of treating schizophrenia comprising administering to a patient in need thereof a therapeutically effective amount of the pharmaceutical formulation of claim 1.
